(19) United States
(12) Patent Application Publication
(10) Pub. No.: US 2025/0243207 A1
(43) Pub. Date: Jul. 31, 2025

(54) SYNTHETIC METHODS FOR PREPARING A PYRIDINECARBOXAMIDE COMPOUND

(71) Applicants: BIOMEA FUSION, INC., Redwood City, CA (US); SHANGHAI SYNTHEALL PHARMACEUTICALS CO., LTD., Shanghai (CN)

(72) Inventors: James T. PALMER, Warrandyte (AU); Thorsten A. KIRSCHBERG, San Carlos, CA (US); Nan-Horng LIN, Vernon Hills, IL (US); Thomas BUTLER, Redwood City, CA (US); Satish Goud PUPPALI, Sunnyvale, CA (US); Heow TAN, Cupertino, CA (US); Bo PENG, Shanghai (CN); Danmei DAI, Shanghai (CN); Binbin SHI, Shanghai (CN); Chunyang HOU, Shanghai (CN)

(21) Appl. No.: 18/993,897

(22) PCT Filed: Jul. 13, 2022
(86) PCT No.: PCT/CN2022/105472

§ 371 Date: Jan. 13, 2025

(51) Int. Cl. C07D 487/04

(52) U.S. Cl. CPC C07D 487/04

(57) ABSTRACT

Described herein are synthetic methods to prepare N-[4-[4-(4-morpholinyl)-7H-pyrrolo[2,3-d]pyrimidin-6-yl]phenyl]-4-[[3-[(1-oxo-2-propen-1-yl) amino]-1-piperidinyl]methyl]-2-pyridinecarboxamide, an irreversible covalent inhibitor of menin-MLL interaction.

### FIELD OF THE INVENTION

Described herein is a preparation of an irreversible inhibitor of menin-MLL N-[4-[4-(4-morpholinyl)-7H-pyrrolo[2,3-d]pyrimidin-6-yl]phenyl]-4-[[3-[(1-oxo-2-propen-1-yl)amino]-1-piperidinyl]methyl]-2-pyridinecarboxamide.

### BACKGROUND OF THE INVENTION

The Histone-lysine N-methyltransferase 2 (KMT2) family of proteins, which currently consists of at least 5 members, methylate lysine 4 on the histone H3 tails at important regulatory regions in the genome and thereby impart crucial functions through the modulation of chromatin structures and DNA accessibility (Morera, Lubbert, and Jung., Clin. Epigenetics 8, 57-(2016)). These enzymes are known to play an important role in the regulation of gene expression during early development and hematopoiesis (Rao & Dou., Nat. Rev. Cancer 15, 334-346 (2015)).

The human KMT2 family was initially named the mixed-lineage leukemia (MLL) family, owing to the role of the first-found member in this disease, KMT2A which is still commonly referred to as MLL1 or MLL in routine clinical practice.

KMT2A (MLL1) is frequently found to be cytogenetically targeted in several types of leukemia (e.g. ALL and AML), and in those cases where balanced chromosomal translocations are found, these typically target KMT2A (MLL1) and one of over 80 translocation partner genes that have been described to date (Winters and Bernt, Front. Pediatr. 5, 4 (2017)). These chromosomal anomalies often result in the formation of fusion genes that encode fusion proteins which are believed to be causally related to the onset and/or progression of the disease. Inhibition of menin may be a promising strategy for treating MLL related diseases, including leukemia.

### SUMMARY OF THE INVENTION

Described herein is a method of preparation for an irreversible inhibitor of menin-MLL interaction N-[4-[4-(4-morpholinyl)-7H-pyrrolo[2,3-d]pyrimidin-6-yl]phenyl]-4-[[3-[(1-oxo-2-propen-1-yl)amino]-1-piperidinyl]methyl]-2-pyridinecarboxamide. Also described are novel heterocyclic compounds as intermediates.

In one particular aspect, described herein is a method for preparation of N-[4-[4-(4-morpholinyl)-7H-pyrrolo[2,3-d]pyrimidin-6-yl]phenyl]-4-[[3-[(1-oxo-2-propen-1-yl)amino]-1-piperidinyl]methyl]-2-pyridinecarboxamide, a compound of Formula I.

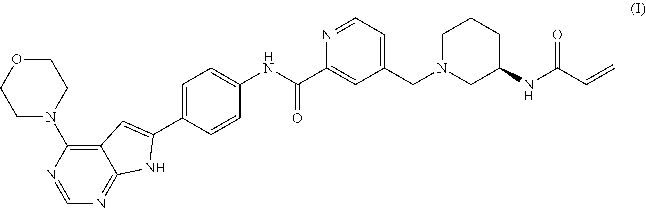 [SMILES: C=CC(=O)N[C@@H]1CCCN(Cc2ccnc(C(=O)Nc3ccc(-c4cc5c(N6CCOCC6)ncnc5[nH]4)cc3)c2)C1]

wherein the method comprises the step of:
   A5) reacting a compound of Formula V:

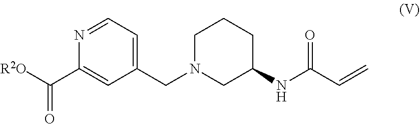

with a compound of Formula VI:

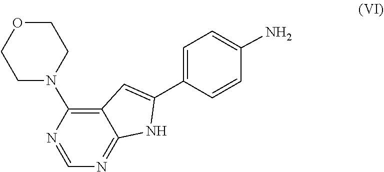 [SMILES: Nc1ccc(-c2cc3c(N4CCOCC4)ncnc3[nH]2)cc1]

to obtain the compound of Formula I; wherein R² is H, Li, Na, K, or Ca.

In another particular aspect, described herein is a method for preparation of a compound of Formula V:

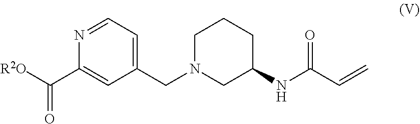

wherein the method comprises the step of:
   A1) providing a compound of Formula II:

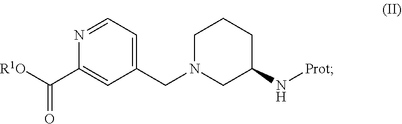

wherein Prot is an amine protecting group, and R¹ is alkyl, or benzyl;
   A2) deprotecting the compound of Formula II to obtain the intermediate compound of Formula III:

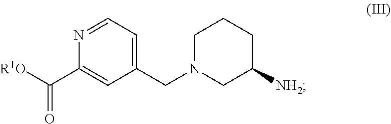

A3) converting the compound of Formula III to the intermediate compound of formula IV:

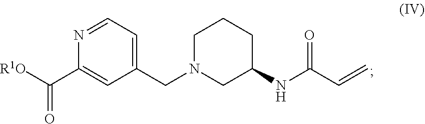

A4) converting the compound of Formula IV to the intermediate compound of formula V:

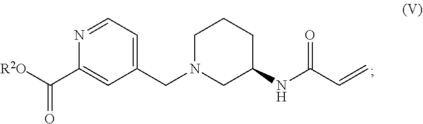

wherein R² is H, Li, Na, K, or Ca.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### INCORPORATION BY REFERENCE

All publications and patent applications mentioned in this specification are herein incorporated by reference to the extent applicable and relevant.

### DETAILED DESCRIPTION OF THE INVENTION

The diverse roles played by menin-MLL interaction in various hematopoietic cell functions suggests that small molecule inhibitors of menin-MLL interaction, such as Compound A, are useful for reducing the risk of or treating a variety of diseases affected by or affecting many cell types of the hematopoetic lineage including, e.g., autoimmune diseases, heteroimmune conditions or diseases, inflammatory diseases, cancer (e.g., B-cell proliferative disorders), and thromboembolic disorders.

Described herein is a method for preparation for an irreversible inhibitor of menin-MLL interaction N-[4-[4-(4-morpholinyl)-7H-pyrrolo[2,3-d]pyrimidin-6-yl]phenyl]-4-[[3-[(1-oxo-2-propen-1-yl)amino]-1-piperidinyl]methyl]-2-pyridinecarboxamide. Also described are novel heterocyclic compounds as intermediates.

In one particular aspect, described herein is a method for preparation of N-[4-[4-(4-morpholinyl)-7H-pyrrolo[2,3-d]pyrimidin-6-yl]phenyl]-4-[[3-[(1-oxo-2-propen-1-yl)amino]-1-piperidinyl]methyl]-2-pyridinecarboxamide, a compound of Formula I:

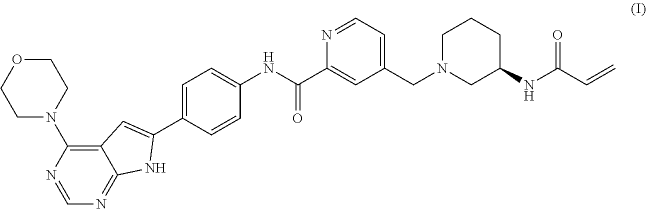 [SMILES: C=CC(=O)N[C@@H]1CCCN(Cc2ccnc(C(=O)Nc3ccc(-c4cc5c(N6CCOCC6)ncnc5[nH]4)cc3)c2)C1]

wherein the method comprises the step of:
   A5) reacting a compound of Formula V:

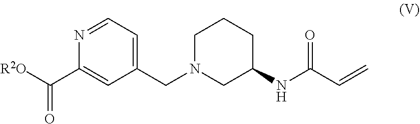

with a compound of Formula VI:

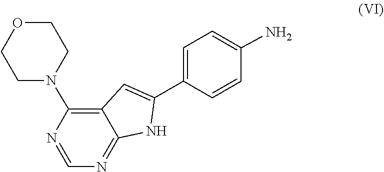 [SMILES: Nc1ccc(-c2cc3c(N4CCOCC4)ncnc3[nH]2)cc1]

to obtain the compound of Formula I; wherein R² is H, Li, Na, K, or Ca.

In certain embodiments, R² is H.

In certain embodiments, R² is Li, Na, K, or Ca.

In certain embodiments, R² is Li.

In certain embodiments, the step A5) is in the absence of solvent.

In certain embodiments, the step A5) is in the presence of solvent.

In certain embodiments, the step A5) is in the presence of a solvent, and the solvent is DMF, DMAc, THF, dioxane, or any other aprotic solvent, or any combination thereof.

In certain embodiments, the step A5) is in DMAc.

In certain embodiments, the step A5) is in the presence of a base.

In certain embodiments, the step A5) is in the presence of a base; and the base is selected from sodium hydride, sodium methoxide, sodium t-butoxide, potassium t-butoxide, potassium carbonate, sodium carbonate, potassium acetate, sodium acetate, trialkylamine, dialkylamine, Hunig's base, DIPEA, N-methyl-morpholine, and any combination thereof.

In certain embodiments, the step A5) is in the presence of DIPEA.

In certain embodiments, the step A5) is in the presence of a coupling agent.

In certain embodiments, the step A5) is in the presence of a coupling agent; and the coupling agent is EDCI, CDI, T3P, TBTU, HCTU, HATU PyBOP, DCC, and any combination thereof.

In certain embodiments, the step A5) is in the presence of EDCI.

In certain embodiments, the step A5) is in the presence of HOPO.

In certain embodiments, the step A5) is in the presence of EDCl and HOPO.

In certain embodiments, the step A5) is in the presence of DIPEA, EDCl, and HOPO

In certain embodiments, the step A5) is at a temperature from about 0° C. to about 100° C. In certain embodiments, the step A5) is at a temperature from about 10° C. to about 60° C. In certain embodiments, the step A5) is at a temperature from about 15° C. to about 40° C. In certain embodiments, the step A5) is at a temperature around 20-40° C. In certain embodiments, the step A5) is at a temperature around 20-30° C., and then at 35-40° C. In certain embodiments, the step A5) is at a temperature from around 25° C.

In certain embodiments, the step A5) is for 1 to 100 hours, 15 to 50 hours, or 20 to 50 hours. In certain embodiments, the step A5) is for 10 to 15 hours

In certain embodiments, the step A5) is for about 30-35 hrs.

In certain embodiments, the intermediate compound of Formula V is prepared using a synthetic process, wherein the process comprises the steps of:
A1) providing a compound of Formula II:

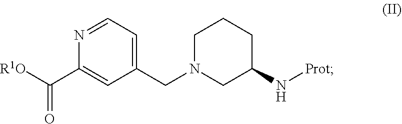

wherein Prot is an amine protecting group, and R¹ is alkyl, or benzyl;
A2) deprotecting the compound of Formula II to obtain the intermediate compound of Formula III:

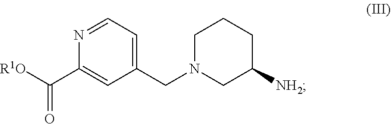

A3) converting the compound of Formula III to the intermediate compound of formula IV:

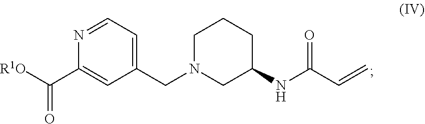

A4) converting the compound of Formula IV to the intermediate compound of formula V:

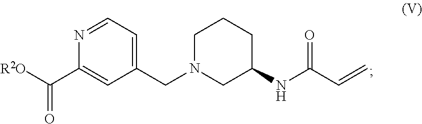

wherein R is H, Li, Na, K, or Ca.

In certain embodiments, R¹ is C₁-C₆ alkyl. In other embodiments, R¹ is C₁-C₄ alkyl.

In certain embodiments, in the step A1) R¹ is Me, Et, i-Pr, or benzyl.

In certain embodiments, in the step A1) R¹ is Me.

In certain embodiments, in the step A1) Prot is Boc.

In certain embodiments, the step A2) is in the absence of solvent.

In certain embodiments, the step A2) is in the presence of solvent.

In certain embodiments, the step A2) is in a solvent, and the solvent is methanol, ethanol, isopropanol, ethyl acetate, dichloromethane, tetrachloroethane, THF, dioxane, or any combination thereof.

In certain embodiments, the step A2) is in methanol.

In certain embodiments, the step A2) is in the presence of an acid.

In certain embodiments, the step A2) is in the presence of an acid; and the acid is selected from methane sulfonic, benzenesulfonic, hydrochloric, hydrobromic, sulfuric, trifluoro acetic acid, TiCl₄, SnCl₄, chiral camphor sulfonic acid, or any combination thereof, and any combination thereof.

In certain embodiments, the step A2) is in the presence of HCl/MeOH.

In certain embodiments, the step A2) is in the presence of 20% HCl/MeOH.

In certain embodiments, the step A2) is at a temperature from about 0° C. to about 100° C. In certain embodiments, the step A2) is at a temperature from about 10° C. to about 50° C. In certain embodiments, the step A2) is at a temperature from about 15° C. to about 40° C.

In certain embodiments, the step A2) is at a temperature between 20-25° C.

In certain embodiments, the step A2) is for 1 to 100 hours, 5 to 50 hours, or 6 to 48 hours.

In certain embodiments, the step A2) is for about 5-15 hrs. In certain embodiments, the step A2) is for around 10 hrs

In certain embodiments, in the step A2) R² is Me.

In certain embodiments, the compound of Formula III is a mono, di, or tri acid salt.

In certain embodiments, the compound of Formula III is a mono, di, or tri acid salt, and the acid salt is a hydrochloric, hydrobromic, methanesulfonic or trifluoroacetic salt.

In certain embodiments, in the step A3) R¹ is Me, Et, i-Pr, or benzyl.

In certain embodiments, in the step A3) R¹ is Me.

In certain embodiments, in the step A3) the conversion is via coupling of the compound of Formula III with acrylic acid, acrylic anhydride, or acryloyl chloride.

In certain embodiments, in the step A3) the conversion is via coupling of the compound of Formula III with acrylic anhydride.

In certain embodiments, the step A3) is in the absence of solvent.

In certain embodiments, the step A3) is in the presence of solvent.

In certain embodiments, the step A3) is in a solvent, and the solvent is DCM, toluene, n-heptane, acetonitrile, THF, dioxane, or any other aprotic solvent, or any combination thereof.

In certain embodiments, the step A3) is in DCM.

In certain embodiments, the step A3) is in the presence of a base.

In certain embodiments, the step A3) is in the presence of a base; and the base is selected from trialkylamine, dialkylamine, alkylamine, Hunig's base, pyridine, imidazole, DIPEA, N-methyl-morpholine, and any combination thereof.

In certain embodiments, the step A3) is in the presence of Hunig's base.

In certain embodiments, the step A3) is at a temperature from about 0° C. to about 100° C.

In certain embodiments, the step A3) is at a temperature around 0-20° C. In certain embodiments, the step A3) is at a temperature around 0-5° C.

In certain embodiments, the step A3) is for 1 to 100 hours, 5 to 50 hours, or 6 to 48 hours.

In certain embodiments, the step A3) is for about 1-5 hrs.

In certain embodiments, in the step A4) R¹ is Me, Et, i-Pr, or benzyl.

In certain embodiments, in the step A4) R¹ is Me.

In certain embodiments, the step A4) is in the absence of solvent.

In certain embodiments, the step A4) is in the presence of solvent.

In certain embodiments, the step A4) is in the presence of a solvent, and the solvent is DMF, DMAc, MeOH, EtOH, iso-PrOH, acetone, THF, dioxane, water, or any combination thereof.

In certain embodiments, the step A4) is in a mixture of DMAc and water.

In certain embodiments, the step A4) is in the presence of a reagent.

In certain embodiments, the step A4) is in the presence of a reagent; and the reagent is selected from LiOH, NaOH, KOH, or Ca(OH)₂.

In certain embodiments, in the step A4) R² is Li; and the reagent is LiOH.

In certain embodiments, in the step A4) R² is Na; and the reagent is NaOH.

In certain embodiments, in the step A4) R² is Na; and the reagent is KOH. In certain embodiments, in the step A4) R² is Ca; and the reagent is Ca(OH)₂.

In certain embodiments, the step A4) is at a temperature from about 0° C. to about 100° C.

In certain embodiments, the step A4) is at a temperature around 20-35° C.

In certain embodiments, the step A4) is for 1 to 100 hours, 5 to 50 hours, or 6 to 48 hours.

In certain embodiments, the step A4) is for about 10-20 hrs.

In certain embodiments, the product, compound of Formula V obtained in step A4) is used directly in step A5). In certain embodiments, the product, compound of Formula V obtained in step A4) is used without isolating in step A5). In certain embodiments, the product, compound of Formula V obtained in step A4) is used without any further purifaction in step A5).

In certain embodiment, the intermediate for synthesis of compound of Formula I, is a compound of Formula X:

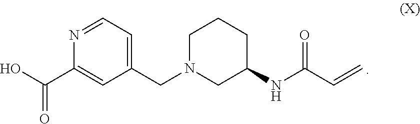 [SMILES: C=CC(=O)N[C@@H]1CCCN(Cc2ccnc(C(=O)O)c2)C1]

In certain embodiment, the intermediate for synthesis of compound of Formula I, is a compound of Formula IV:

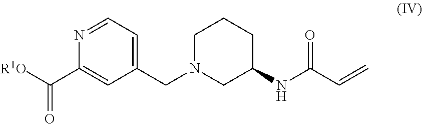

and wherein R¹ is Me, Et, n-Pr, i-Pr, n-Bu, iso-Bu, sec-Bu, or t-Bu.

In certain embodiment, the intermediate for synthesis of compound of Formula I, is a compound of Formula III:

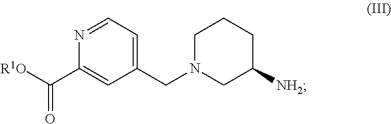

and wherein R¹ is Me, Et, n-Pr, i-Pr, n-Bu, iso-Bu, sec-Bu, or t-Bu.

In certain embodiments, R¹ is Me, or Et.

In certain embodiments, R¹ is Me.

In certain embodiment, the intermediate for synthesis of compound of Formula I, is a compound of Formula XI:

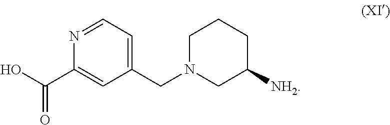 [SMILES: N[C@@H]1CCCN(Cc2ccnc(C(=O)O)c2)C1]

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

‘Alkyl’ means straight or branched aliphatic hydrocarbon having 1 to 20 carbon atoms. Particular alkyl has 1 to 12 carbon atoms. More particular is lower alkyl which has 1 to 6 carbon atoms. A further particular group has 1 to 4 carbon atoms. Exemplary straight chained groups include methyl, ethyl n-propyl, and n-butyl. Branched means that one or more lower alkyl groups such as methyl, ethyl, propyl or butyl is attached to a linear alkyl chain, exemplary branched chain groups include isopropyl, iso-butyl, t-butyl and isoamyl.

The term “identical,” as used herein, refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using comparison algorithms or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exists over a region that is at least about 75-100 sequential units in length, over a region that is about 50 sequential units in length, or, where not specified, across the entire sequence. This definition also refers to the complement of a test sequence. By way of example only, two or more polypeptide sequences are identical when the amino acid residues are the same, while two or more polypeptide sequences are “substantially identical” if the amino acid residues are about 60% identical, about 65% identical, about 70% identical, about 75% identical, about 80% identical, about 85% identical, about 90% identical, or about 95% identical over a specified region. The identity can exist over a region that is at least about 75-100 amino acids in length, over a region that is about 50 amino acids in length, or, where not specified, across the entire sequence of a polypeptide sequence. In addition, by way of example only, two or more polynucleotide sequences are identical when the nucleic acid residues are the same, while two or more polynucleotide sequences are “substantially identical” if the nucleic acid residues are about 60% identical, about 65% identical, about 70% identical, about 75% identical, about 80% identical, about 85% identical, about 90% identical, or about 95% identical over a specified region. The identity can exist over a region that is at least about 75-100 nucleic acids in length, over a region that is about 50 nucleic acids in length, or, where not specified, across the entire sequence of a polynucleotide sequence.

The terms “inhibits”, “inhibiting”, or “inhibitor” of a menin, as used herein, refer to inhibition of menin activity, for instance menin-MLL interaction and activity.

The term “irreversible inhibitor,” as used herein, refers to a compound that, upon contact with a target protein (e.g., menin or menin-HLL) causes the formation of a new covalent bond with or within the protein, whereby one or more of the target protein's biological activities (e.g., phosphotransferase activity) is diminished or abolished notwithstanding the subsequent presence or absence of the irreversible inhibitor.

The term “irreversible menin inhibitor,” as used herein, refers to an inhibitor of menin that can form a covalent bond with an amino acid residue of menin.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed ‘diastereomers’ and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a ‘racemic mixture’.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 80% by weight R-compound and at most about 20% by weight S-compound, at least about 90% by weight R-compound and at most about 10% by weight S-compound, at least about 95% by weight R-compound and at most about 5% by weight S-compound, at least about 99% by weight R-compound and at most about 1% by weight S-compound, at least about 99.9% by weight R-compound or at most about 0.1% by weight S-compound. In certain embodiments, the weights are based upon total weight of compound.

As used herein and unless otherwise indicated, the term “enantiomerically pure S-compound” or “S-compound” refers to at least about 80% by weight S-compound and at most about 20% by weight R-compound, at least about 90% by weight S-compound and at most about 10% by weight R-compound, at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art

### EXAMPLES

The following ingredients, formulations, processes and procedures for practicing the methods disclosed herein correspond to that described above.

The compounds can be prepared from readily available starting materials using the following methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds can be isolated and purified by known standard procedures. Such procedures include (but are not limited to) recrystallization, column chromatography or HPLC. The following schemes are presented with details as to the preparation of representative fused heterocyclics that have been listed hereinabove. The compounds may be prepared from known or commercially available starting materials and reagents by one skilled in the art of organic synthesis.

The compounds of the present invention may be prepared by procedures described herein.

In this specification, the following abbreviations can be used:
Ad₂(n-Bu)P Di(1-Adamantyl)-n-butylphophine
BEP 2-Bromo-1-ethylpyridinium tetrafluoroborate
BOP Benzotriazol-1-yloxy-tris(dimethylamino)phosphonium hexafluorophosphate
CDI 1,1′-Carbonyldiimidazole
DCC N,N′-Dicyclohexylcarbodiimide
DCM Dichloromethane
DIPEA N,N′-Diisopropylethylamine, Hunig's base
DMAc N,N′-Dimethylacetamide
DME 1,2-Dimethoxyethane, Dimethoxyethane
DMF N,N-Dimethylformamide
DMSO Dimethyl sulfoxide
EDAC·HCl 1-Ethyl-3-(3′-dimethylaminopropyl)carbodiimide Hydrochloride
EDAC 1-Ethyl-3-(3′-dimethylaminopropyl)carbodiimide
EtOAc Ethyl acetate
EtOH Ethanol
HATU 1-[Bis(dimethylamino)methylene]-1H-1,2,3-triazolo[4,5-b]pyridinium 3-oxide hexafluorophosphate
HCTU 2-(6-Chloro-1H-benzotriazole-1-yl)-1,1,3,3-tetramethylaminium hexafluorophosphate
HOBt 1-Hydroxybenzotriazole
HOPO 2-Hydroxypyridine-N-oxide, or 2-Pyridinol-1-oxide
MeOH Methanol
NMP N-Methyl-2-pyrrolidone
Pd(OAc)₂ Palladium acetate
PyBOP Benzotriazol-1-yl-oxy-tris-pyrrolidinophosphonium hexafluorophosphate
T3P Propylphosphonic anhydride
TBTU 1-[Bis(dimethylamino)methylene]-1H-benzotriazolium 3-Oxide Tetrafluoroborate
THF Tetrahydrofuran
TFA Trifluoroacetic acid
μM Micro Molar
μL Micro Liter

### Example 1

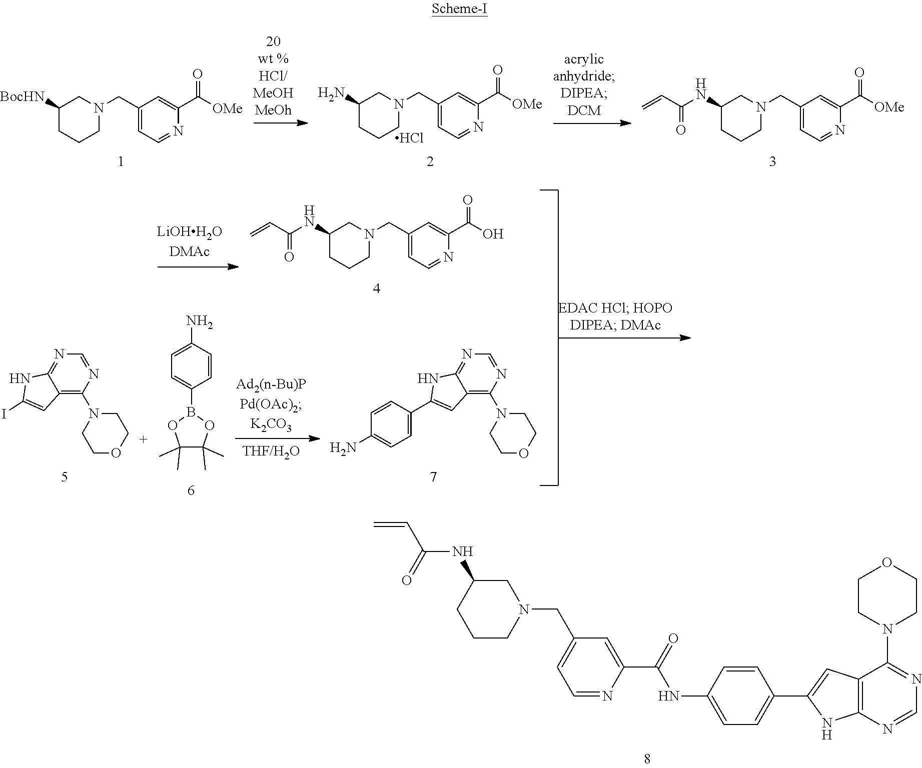

### Synthesis of Methyl (R)-4-((3-aminopiperidin-1-yl) methyl) picolinate·HCl (2)

Compound-1 (5.0 g, 2.86 mmol) was dissolved in Methanol (40 mL) and the mixture was cooled to 0-5° C. Methanolic-HCl solution (3.0M, 15 mL, 9.0 mmol) was added slowly over a period of 15 min while maintaining the temp. between 0-5° C. The mixture was allowed to warm to room temperature and stirred for about 10 h. Methanol was concentrated under vacuum to obtain the residue. Ethyl acetate (25 mL) was added to the residue and concentrated to 10 mL. The mixture was cooled to 0-5° C., filtered, and dried in a vacuum oven at 40-50° C. Weight: 3.2 g; Yield: 90%. ¹H-NMR (CDCl₃): δ ppm 12.27 (br s, 1H), 8.79 (d, J=4.82 Hz, 1H), 8.67 (br s, 3H), 8.33 (s, 1H), 8.01 (br d, J=4.39 Hz, 1H), 4.46-4.59 (m, 2H), 3.87 (s, 3H), 3.60 (br s, 1H), 3.41 (br d, J=9.87 Hz, 1H), 3.30 (br d, J=10.30 Hz, 1H), 3.12 (d, J=0.66 Hz, 1H), 2.97 (br t, J=11.18 Hz, 1H), 2.86 (br s, 1H), 2.06 (br d, J=10.52 Hz, 1H), 1.79-1.98 (m, 2H), 1.45-1.63 (m, 1H). M⁺¹: 250.47.

### Synthesis of Methyl (R)-4-((3-acrylamidopiperidin-1-yl)methyl)picolinate (3)

Crude Methyl (R)-4-((3-aminopiperidin-1-yl)methyl)picolinate·HCl (2, 3.0 g, 10.5 mmol) was suspended in Dichloromethane (15 mL) and the mixture was cooled to 0-5° C. Hunig's base (4.2 mL, 24.0 mmol) was added slowly and then the mixture was stirred for 15 min. Acrylic anhydride (1.46 g, 11.57 mmol) was added slowly through syringe while maintaining the temp below 0° C. The mixture was stirred for 3.0 h, and the pH of the mixture was adjusted to 7.0 with Citric acid solution (5%). The mixture was slowly warmed to room temperature and then stirred for 1.0 h and transferred into a separating funnel. The DCM layer was separated, and the aqueous layer was extracted with DCM (20 ML) and separated. The combined DCM layer was separated and washed with 10% Na₂SO₄ solution. The DCM layer was concentrated under vacuum and triturated with Toluene to obtain Compound-3. Weight: 2.0 g; Yield: 82%. ¹H-NMR (CDCl₃): δ ppm 8.57 (d, J=5.04 Hz, 1H), 8.13 (s, 1H), 7.62 (dd, J=4.93, 1.21 Hz, 1H), 6.11-6.30 (m, 2H), 5.61 (dd, J=9.76, 2.30 Hz, 1H), 3.89-4.03 (m, 4H), 3.62 (s, 2H), 2.81 (br d, J=8.99 Hz, 1H), 2.56-2.69 (m, 1H), 2.17 (br t, J=9.54 Hz, 1H), 2.05 (br t, J=9.54 Hz, 1H), 1.70-1.88 (m, 2H), 1.55-1.69 (m, 1H), 1.27-1.40 (m, 2H). M⁺¹: 304.18

### Synthesis of (R)-4-((3-acrylamidopiperidin-1-yl)methyl)picolinic acid (4)

Compound-3 (5.0 g, 16.48 mmol) was dissolved in a mixture of N,N′-Dimethylacetamide (DMAc, 25 mL) an DI water (0.5 mL). To the clear solution, Lithium hydroxide (0.83 g, 34.6 mmol) was added in small portions at room temperature and stirred for 20 h at 25-35° C. After completion of hydrolysis, the mixture was cooled to 5-10° C. and the pH of the mixture was adjusted to ˜7.0 with 10% H₂SO₄-DMAc solution while maintaining the temp. below 10° C. The mixture was concentrated under vacuum, followed by azeotropic distillation with Toluene to obtain Compound-4 as DMAc-solution, which was used for the next coupling reaction without any purification.

### Synthesis of 4-(4-morpholino-7h-pyrrolo[2,3-d]pyrimidin-6-yl)aniline (7) (U.S. Pat. No. 1,084,825) (Incorporated by Reference in its Entirety)

Potassium carbonate (6.2 g, 45 mmol) DI Water (15 mL), THE (65 mL) were placed in a round bottom flask and bubbled the mixture with N₂ for 10 min. Pd(OAc)₂ (0.117 g, 0.52 mmol), and Ad₂(n-Bu)P (0.322 g, 0.90 mmol) were place into the round bottom flask and a stream of N₂ was bubbled for 10 min. Under N₂ atmosphere, Compound-5 (5.0 g, 15.15 mmol) and Compound-6 (3.8 g, 17.35 mmol) were placed and bubbled the N₂ for 10 min. The reaction mixture was slowly heated to 60-70° C. and stirred for 40 h. After the reaction was completed, THE was concentrated under vacuum and quenched the mixture with DI water (100 mL). Stir the mixture for additional 10 h at RT and filtered the precipitated solid to obtain Compound-7. Weight: 3.20 g; Yield: 72%. ¹H-NMR (CDCl₄): δ12.27 (br s, 1H), 8.08 (s, 1H), 6.88 (s, 1H), 3.80-3.77 (m, 4H), 3.70-3.68 (m, 4H). M⁺¹: 296.58.

### Synthesis of (R)-4-((3-Acrylamidopiperidin-1-yl) methyl)-N-(4-(4-morpholino-7H-pyrrolo[2,3-d]pyrimidine-6-yl)phenyl)picolinamide (8)

Compound-4 (12 g, 41.4 mmol, Assay corrected), N,N′-Dimethylacetamide (10 mL) were placed in a round bottom flask and arranged for stirring at room temperature. EDAC·HCl (13.03 g, 68 mmol), 2-Pyridinol-1-oxide (7.5 g, 67.5 mmol), and N,N-Diisopropylethylamine (17.57 g, 136 mmol) were added sequentially while maintaining the temperature below 25° C. After the mixture was stirred for 0.5 h, Compound-7 (10.0 g, 34 mmol, dissolved in 40 mL of N,N′-DMAc) was added slowly over a period of 1.0 h while maintaining the temperature below 25° C. The reaction mixture was stirred for overnight at 35-40° C. and quenched with water (100 mL) at RT. The precipitated solid was filtered, washed with water (50 mL) and dried in a vacuum oven to obtain the pure compound. Weight: 13.40 g, Yield: 70%. ¹H-NMR (DMSO): δ ppm 12.21 (s, 1H), 10.74 (s, 1H), 8.69 (d, J=5.01 Hz, 1H), 8.19 (s, 1H), 8.12 (s, 1H), 7.96-8.04 (m, 3H), 7.81-7.94 (m, 2H), 7.63 (d, J=4.53 Hz, 1H), 7.17 (d, J=1.43 Hz, 1H), 6.17-6.29 (m, 1H), 6.00-6.11 (m, 1H), 5.47-5.63 (m, 1H), 3.80-3.98 (m, 5H), 3.70-3.80 (m, 4H), 3.66 (s, 2H), 2.79 (br d, J=7.99 Hz, 1H), 2.66 (br d, J=11.68 Hz, 1H), 1.97-2.14 (m, 1H), 2.12 (s, 1H), 1.82-1.97 (m, 1H), 1.64-1.83 (m, 2H), 1.46-1.60 (m, 1H), 1.10-1.31 (m, 1H). M⁺¹: 567.44

### Synthesis of Compound-4 (U.S. Pat. No. 11,084,825) (Incorporated by Reference in its Entirety)

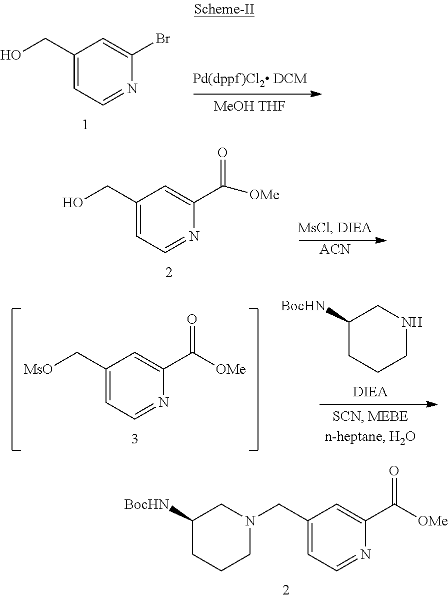

### Synthesis of 2-Bromopyridin-4yl-methanol (1)

To a solution of compound-1 (5.0 g, 26.60 mmol), Triethylamine (9.42 g, 93 mmol, 3.50 mmol), in Methanol (20 mL) was added Pd(dppf)C₂·CH₂Cl₂ (0.10 g, 0.12 mmol, 0.005 mmol) under N₂ atmosphere. The suspension was degassed and purged with CO for 3 times. The mixture was stirred under CO (2 MPa) at 80° C. for 12 h. After the mixture was completed, the mixture was filtered and concentrated under vacuum to obtain the crude solid. The crude product was triturated with DME (15 mL) and then filtered to obtain the compound 2 as a pink solid. Weight: 3.0 g, Yield: 69%. ¹H-NMR (CDCl₃): 8.54 (d, J=4.88 Hz, 1H), 8.03 (s, 1H), 7.44 (br d, J=4.88 Hz, 1H), 4.75 (s, 2H), 3.80-4.03 (m, 4H). M⁺¹: 168.47.

### Synthesis of Methyl-4-(methyl sulfonyl)oxy)methyl)picolinate (2)

To a solution of compound-2 (10 g, 59.82 mmol) and N′N′-Diisopropylamino ethylamine (15.46 g, 120 mmol) in dichloromethane (50 mL) was added Methane sulphonyl chloride (10.27 g, 89.73 mmol) at 0-5° C. The mixture was stirred at 0-5° C. for 2.0 h. After completion of reaction, the mixture was quenched with water (20 mL) and then extracted with DCM (2×25 mL). The combined DCM layer was washed with water, brine, and dried over sodium sulfate. The DCM layer was filtered, and concentrated under vacuum to obtain the residue, which was triturated with n-Heptane (25 mL). The precipitated solid was filtered and dried. Weight: 9.8 g, Yield: 67%. ¹H-NMR (CDCl₃): δ ppm 8.74 (d, J=4.85 Hz, 1H), 8.10 (s, 1H), 7.49 (d, J=4.19 Hz, 1H), 5.27 (s, 2H), 3.91-4.04 (m, 3H), 3.00-3.11 (m, 3H). M⁺¹: 246.37

### Synthesis of Methyl-(R)-4-(3-tert-butoxycarbonyl) amino) piperidine-1-yl) methyl) picolinate (4)

To a solution of compound-3 (10 g, 40.7 mmol), and tert-butyl-(R)-piperidine-3-yl-carboxylate (8.57 g, 42.8 mmol) was added anhydrous Potassium carbonate (17 g, 123 mmol) at 20° C. The mixture was slowly heated to 100° C. and then maintained for 12 h. The mixture was cooled to room temperature and quenched with water (60 mL). The mixture was extracted with Ethyl acetate (2×30 mL) and then concentrated under vacuum and the resultant residue was triturated with n-Heptane (25 mL) to obtain pale yellow solid. Weight: 10.5 g, Yield: 74%.

¹H-NMR (CDCl₃): δ ppm 8.61 (d, J=4.88 Hz, 1H), 8.00 (s, 1H), 7.41 (br d, J=4.38 Hz, 1H), 4.84 (br s, 1H), 3.87-4.06 (m, 3H), 3.57-3.80 (m, 1H), 3.47 (s, 2H), 2.54 (br d, J=8.88 Hz, 1H), 2.26 (br d, J=15.13 Hz, 3H), 1.62 (br s, 2H), 1.46-1.55 (m, 1H), 1.37 (s, 10H). M⁺¹:350.24.

### Synthesis of Compound-7

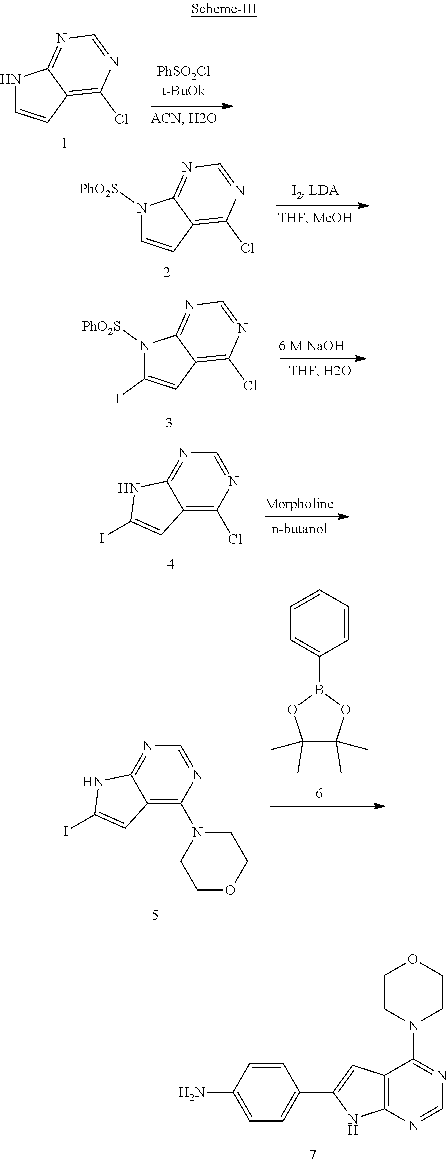

### Synthesis of 4-Chloro-7-(phenylsulfonyl)-7H-pyrrolo[2,3-D]pyrimidine (2)

To a solution of compound 1 (10 g, 65.1 mmol) in THF (100 mL) was added t-BuONa (7.67 g, 68.37 mmol), and then PhSO₂Cl (11.50 g, 65.1 mol) was added dropwise at 10° C. The resulting mixture was stirred at 20° C. for 5 hrs. TLC (Petroleum ether/Ethyl acetate=3/1, R_{f}=0.51) showed the reaction was complete. The reaction mixture was quenched with DI Water (50 mL), filtered the precipitated solid, washed with MeOH (20×2), and dried in a vacuum oven. Weight: 17.4 g, Yield: 91%. ¹H-NMR (DMSO): δ 8.81 (s, 1H), 8.17-8.12 (m, 3H), 7.77-7.75 (m, 1H), 7.68-7.64 (m, 2H), 6.95 (d, J=4.0 Hz, 1H). M⁺¹: 294.79

### Synthesis of 4-Chloro-7-(phenylsulfonyl)-7H-pyrrolo[2,3-d]pyrimidine (3)

To a solution of compound 2 (10 g, 34 mmol) in THE (75 mL) was added dropwise LDA (2 M, 25.5 mL) at −60° C. After addition, the mixture was stirred at this temperature for 1 h, and then I2 (11.23 g, 44.25 mol) in THE (25 mL) was added dropwise at −60° C. The resulting mixture was stirred at −65° C. for 12 hrs. After the reaction was completed, the mixture was quenched with 1M HCl (50 mL) at 0° C. The organic layer was concentrated under reduced pressure to obtain a residue, which was triturated with MTBE. The precipitated solid was filtered, washed with MTBE and dried in a vacuum oven. Weight: 6.3 g, Yield: 44%.

¹H-NMR (DMSO): δ 8.76 (s, 1H), 8.11-8.09 (m, 2H), 7.82-7.76 (m, 1H), 7.77-7.69 (m, 2H), 7.37 (s, 1H). M⁺¹: 420.17

### Synthesis of 4-Chloro-6-iodo-7H-pyrrolo[2,3-d]pyrimidine (4)

To a solution of compound 3 (8.0 g 19.06 mmol) in THE (80 mL) was added NaOH (2M, 20 mL). The mixture was stirred at 25° C. for 12 hrs. After the reaction was completed, THE was concentrated under reduced pressure, and the resulting mixture pH was adjusted to pH: 7.0 with 2M HCl. The precipitated solid was filtered and washed with water and dried in a vacuum oven to obtain as an off-white solid. Weight: 4.68 g. Yield: 88%.

¹H-NMR (DMSO): δ13.13 (br s, 1H), 8.51 (s, 1H), 6.87 (d, J=2.0 Hz, 1H). M⁺¹: 280.32

### Synthesis of 4-(6-iodo-7H-pyrrolo[2,3-d]pyrimidin-4-yl)morpholine (5)

To a solution of compound 4 (15 g, 53.67 mmol) in n-butanol (80 mL) was added Morpholine (9.33 g, 107 mmol). The mixture was stirred at 100° C. for 12 hrs. After the reaction was completed, the mixture was cooled to 10-15° C. The resulting solid was filtered and washed with cold n-butanol to obtain compound 5 as a white solid. Weight: 13.8 g, yield: 78%. ¹H-NMR (DMSO): δ12.27 (br s, 1H), 8.08 (s, 1H), 6.88 (s, 1H), 3.80-3.77 (m, 4H), 3.70-3.68 (m, 4H). M⁺¹: 331.18

### Synthesis of 4-(4-Morpholino-7H-pyrrolo[2,3-d]pyrimidine-4-yl)morpholine (6)

To a solution of Compound 5 (12 g, 36.3 mmol) and 4-Aminophenylboronic acid pinacol ester (9.46 g, 43.2 mmol) in dioxane (90 mL) and H₂O (20 mL) was added Pd(dppf)Cl₂ (0.52 g, 0.72 mmol) and K₂CO₃ (10 kg, 72 mmol) under N₂. The mixture was stirred at 100° C. for 12 hrs. After the reaction was completed, the mixture was cooled to room temperature and additional water (200 mL) was added slowly over a period of 30 min. The precipitated solid was filtered washed with excess of water and dried in a vacuum oven for 24 h at 55° C. Weight: 7.3 g. Yield: 68%. ¹H-NMR (DMSO): δ 11.92 (s, 1H), 8.12 (s, 1H), 7.58-7.55 (m, 2H), 6.82 (s, 1H), 6.60-6.58 (m, 2H), 5.29 (s, 2H), 3.84-3.82 (m, 4H), 3.74-3.72 (m, 4H). M⁺¹: 296.12.

The examples and embodiments described herein are illustrative and various modifications or changes suggested to persons skilled in the art are to be included within this disclosure. As will be appreciated by those skilled in the art, the specific components listed in the above examples may be replaced with other functionally equivalent components, e.g., diluents, binders, lubricants, fillers, and the like.

1. A method for preparation of a compound of Formula I: 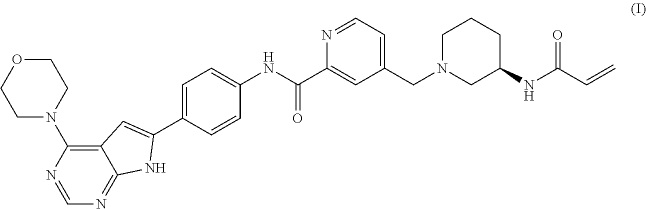 [SMILES: C=CC(=O)N[C@@H]1CCCN(Cc2ccnc(C(=O)Nc3ccc(-c4cc5c(N6CCOCC6)ncnc5[nH]4)cc3)c2)C1]
wherein the method comprises the step of:
A5) reacting a compound of Formula V: 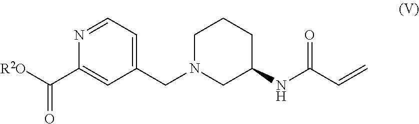
with a compound of Formula VI: 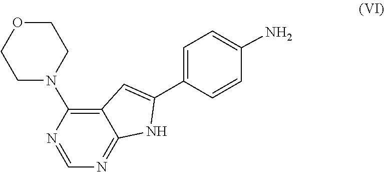 [SMILES: Nc1ccc(-c2cc3c(N4CCOCC4)ncnc3[nH]2)cc1]
to obtain the compound of Formula I; wherein R² is H, Li, Na, K, or Ca.

2.-4. (canceled)

5. The method of claim 1, wherein the method comprises one or more of the following:
the step A5) is in the absence of solvent or in the presence of solvent;
the step A5) is in the presence of a base; and
the step A5) is in the presence of a coupling agent.

6. (canceled)

7. The method of claim 1, wherein the method comprises one or more of the following:
the step A5) is in the presence of a solvent, and the solvent is DMF, DMAc, THF, dioxane, or any other aprotic solvent, or any combination thereof;
the step A5) is in the presence of a base; and the base is selected from sodium hydride, sodium methoxide, sodium t-butoxide, potassium t-butoxide, potassium carbonate, sodium carbonate, potassium acetate, sodium acetate, trialkylamine, dialkylamine, Hünig's base, DIPEA, N-methyl-morpholine, or any combination thereof; and
the step A5) is in the presence of a coupling agent; and the coupling agent is EDCI, CDI, T3P, TBTU, HCTU, HATU PyBOP, DCC, HOPO, or any combination thereof.

8.-21. (canceled)

22. The method of claim 1, wherein the intermediate compound of Formula V is prepared using a synthetic process, wherein the process comprises the steps of:
A1) providing a compound of Formula II: 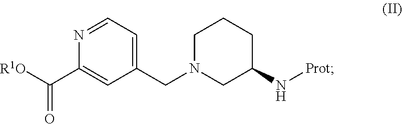
wherein Prot is an amine protecting group, and R¹ is alkyl, or benzyl;
A2) deprotecting the compound of Formula II to obtain the intermediate compound of Formula III: 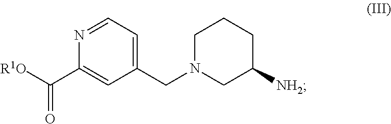
A3) converting the compound of Formula III to the intermediate compound of formula IV: 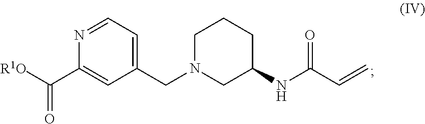
A4) converting the compound of Formula IV to the intermediate compound of formula V: 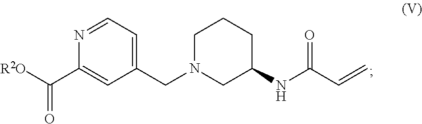
and wherein R² is H, Li, Na, K, or Ca.

23. The method of claim 22, wherein the method comprises one or more of the following:
in the step A1) R¹ is Me, Et, i-Pr, or benzyl; or in the step A1) Prot is Boc; or in the step A1) R¹ is Me, Et, i-Pr, or benzyl, and Prot is Boc;
the step A2) is in the absence of solvent or in the presence of solvent;
the step A2) is in the presence of an acid;
the step A3) is in the absence of solvent or in the presence of solvent;
the step A3) is in the presence of a base;
the step A4) is in the absence of solvent or in the presence of solvent; and
the step A4) is in the presence of a reagent.

24.-27. (canceled)

28. The method of claim 22, wherein the method comprises one or more of the following:
the step A2) is in the presence of a solvent, and the solvent is methanol, ethanol, isopropanol, ethyl acetate, dichloromethane, tetrachloroethane, THF, dioxane, or any combination thereof;
the step A2) is in the presence of an acid; and the acid is selected from methane sulfonic, benzenesulfonic, hydrochloric, hydrobromic, sulfuric, trifluoro acetic acid, TiCl₄, SnCl₄, chiral camphor sulfonic acid, or any combination thereof; and
the step A2) is at a temperature from about 0° C. to about 100° C.

29.-38. (canceled)

39. The method of claim 22, wherein the compound of Formula III is a mono, di, or tri acid salt.

40. The method of claim 22, wherein the compound of Formula III is a mono, di, or tri acid salt, and the acid salt is a hydrochloric, hydrobromic, methanesulfonic or trifluoroacetic salt.

41.-42. (canceled)

43. The method of claim 22, wherein in the step A3) the conversion is via coupling of the compound of Formula III with acrylic acid, acrylic anhydride, or acryloyl chloride.

44.-46. (canceled)

47. The method of claim 22, wherein the method comprises one or more of the following:
the step A3) is in a solvent, and the solvent is DCM, toluene, n-heptane, acetonitrile, THF, dioxane, or any other aprotic solvent, or any combination thereof;
the step A3) is in the presence of a base; and the base is selected from trialkylamine, dialkylamine, alkylamine, Hünig's base, pyridine, imidazole, DIPEA, N-methyl-morpholine, or any combination thereof;
the step A4) R¹ is Me, Et, i-Pr, or benzyl;
the step A4) is in the presence of a solvent, and the solvent is DMF, DMAc, MeOH, EtOH, iso-PrOH, acetone, THF, dioxane, water, or any combination thereof; and
the step A4) is in the presence of a reagent; and the reagent is selected from LiOH, NaOH, KOH, or Ca(OH)₂.

48.-72. (canceled)

73. The method of claim 1, wherein the product, compound of Formula V obtained in step A4) is used without any further purification in step A5).

74. A compound according to one of the following: 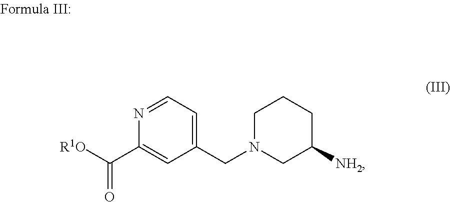
wherein R¹ is Me, Et, n-Pr, i-Pr, n-Bu, iso-Bu, sec-Bu, or t-Bu; 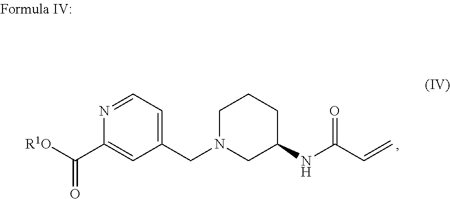
wherein R¹ is Me, Et, n-Pr, i-Pr, n-Bu, iso-Bu, sec-Bu, or t-Bu;

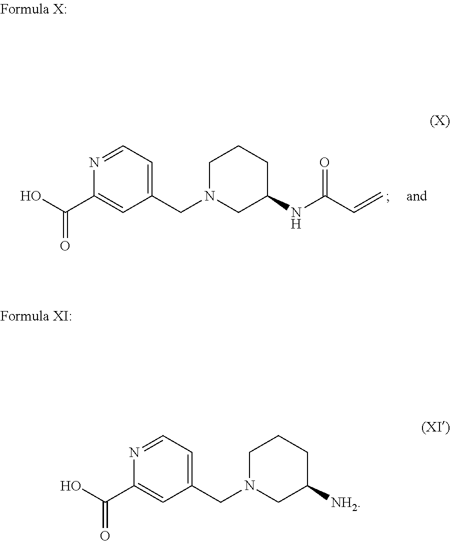 [SMILES: C=CC(=O)N[C@@H]1CCCN(Cc2ccnc(C(=O)O)c2)C1 ; N[C@@H]1CCCN(Cc2ccnc(C(=O)O)c2)C1]

75.-79. (canceled)
